(19) United States
(12) Patent Application Publication
(10) Pub. No.: US 2025/0239347 A1
(43) Pub. Date: Jul. 24, 2025

(54) APPARATUS AND METHOD FOR CONTROLLING PHARMACEUTICAL MIXER OF ADHD MEDICATION BY ASSESSING MENTAL HEALTH OF ADOLESCENT THROUGH ARTIFICIAL INTELLIGENCE

(71) Applicants: LumanLab Inc., Sejong-si (KR); Korea University Research and Business Foundation, Seoul (KR); Korea University Research and Business Foundation, Sejong Campus, Sejong-si (KR)

(72) Inventors: Jae Hyun LIM, Seoul (KR); Chul Hyun CHO, Seoul (KR); Seung Pil PACK, Sejong-si (KR)

(21) Appl. No.: 19/080,808

(22) Filed: Mar. 15, 2025

Related U.S. Application Data
(63) Continuation-in-part of application No. 18/544,488, filed on Dec. 19, 2023.

(30) Foreign Application Priority Data
Dec. 23, 2022 (KR) 10-2022-0183164

(51) Int. Cl. G16H 20/10; A61B 5/00; A61B 5/16; A61B 5/372; G16H 10/20; G16H 10/60

(52) U.S. Cl. CPC G16H 20/10; A61B 5/168; A61B 5/372; A61B 5/7275; A61B 5/742; A61B 5/7455; A61B 5/7475; G16H 10/20; G16H 10/60

(57) ABSTRACT

An apparatus and a method for controlling a pharmaceutical mixer of a medication for Attention Deficit Hyperactivity Disorder (ADHD) based on an assessment of a mental health state of an adolescent. The method comprises: collecting physical information of a user; verifying survey questions regarding mental health depending on the physical information and real-time brain activity data; forming additional survey questions after having verified the answers and calculating a prediction rate of an appearance of symptoms of a mental illness by using AI models; dynamically selecting the most suitable AI model for mental health assessment; verifying a set of survey questions including a plurality of sub-questions depending on the calculated prediction rate; and; outputting result data by adjusting the prediction rate; and transmitting a control signal to the pharmaceutical mixer for the ADHD medicine based on the assessment of the mental health state.

### CROSS REFERENCE TO RELATED APPLICATION

This is a continuation-in-part application claiming priority to U.S. non-provisional application Ser. No. 18/544,488 filed on Dec. 19, 2023 claiming priority to Korea Patent Application No. 10-2022-0183164 filed on Dec. 23, 2022, which is hereby incorporated by reference in its entirety.

### BACKGROUND

### 1. Field of Technology

The present disclosure relates to an apparatus and a method for controlling a pharmaceutical mixer of a medication for Attention Deficit Hyperactivity Disorder (ADHD) based on an assessment of a mental health state of an adolescent.

Also, the present disclosure relates to an electronic device of a service platform system for predicting and managing the revelation of psychosomatic symptoms by periodically assessing the mental health of a school-aged adolescent through an analysis of answers to a survey based on an artificial intelligence (AI), in combination with real-time neurophysiological data obtained via an EEG (electroencephalogram) sensor, and a method therefor.

More particularly, the present disclosure relates to a hybrid assessment system that integrates a structured AI-driven survey method with neurophysiological monitoring, thereby enabling more accurate and dynamic mental health predictions. The system utilizes an EEG sensor for collecting real-time brain activity data while the adolescent is interacting with the AI-based survey. The collected EEG signals, along with survey responses, are processed by an AI model to refine mental health assessments and dynamically adjust the survey based on the user's cognitive and emotional state.

### 2. Related Technology

Generally, a diagnosis of a mental illness of a school-aged adolescent may be confirmed through counseling with a psychiatrist and, depending on the result, a non-drug treatment or a drug treatment may be conducted.

Since communication technologies and digital health care innovations have advanced, psychiatric practices now incorporate standardized mental health surveys administered via mobile devices or digital platforms. Furthermore, the increasing adoption of telemedicine and AI-assisted healthcare services allows patients to seek mental health consultations remotely through applications and web services.

Nevertheless, existing mental health assessments primarily rely on self-reported surveys, which are inherently subjective and susceptible to biases. Additionally, due to negative social perceptions surrounding psychological counseling and the continued necessity for direct psychiatrist observations in offline medical settings, many individuals avoid or delay seeking professional mental health support.

Traditionally, a mental health diagnosis for an adolescent begins when their guardian arranges an in-person visit to a psychiatrist's office, during which the guardian completes a survey regarding a specific mental illness. The psychiatrist then verifies the responses and makes an evaluation based on observational analysis and the provided answers.

However, this process presents multiple challenges: (1) guardians must coordinate appointments and visit medical institutions, (2) guardians must complete various surveys, and (3) survey responses are often influenced by guardians over-interpreting the adolescent's behavior. Additionally, while mobile applications and web-based mental health screening tools have emerged, these solutions lack neurophysiological validation and comprehensive psychiatric oversight, reducing their clinical reliability.

The discussions in this section are only to provide background information and do not constitute an admission of prior art.

### SUMMARY

The present disclosure is to automatically control a pharmaceutical mixer for the ADHD medication based on an assessment of a mental health state of an adolescent. Also, the present disclosure is to assist a diagnosis of a psychiatrist by analyzing, through a means of a machine learning model, bioelectrical activity data collected by an EEG sensor along with responses to survey questions that an adolescent may easily answer by themselves through a mobile device or a digital device; the EEG sensor measures brainwave activity and cognitive responses, which are analyzed in conjunction with survey answers to improve the accuracy of mental health assessments; by integrating both physiological and behavioral data, the system predicts a mental health state of the adolescent or the possibility of an occurrence of a symptom of a mental illness and presents the assessment results to a guardian or a psychiatrist.

An electronic device according to an embodiment, to assess a mental health state of an adolescent by using an EEG sensor and a survey formed based on an artificial intelligence, may comprise a question collecting module to collect physical information of a user through a pre-installed application, to verify survey questions regarding mental health depending on the physical information of the user, and to verify answers to the verified survey questions inputted by the user through the application; an AI model module to form additional survey questions for the user after having verified the answers and to calculate a prediction rate of an appearance of symptoms of a mental illness regarding the additional survey questions by using AI models; a branching recommending module to verify a set of survey questions including a plurality of sub-questions depending on the calculated prediction rate of an appearance of symptoms of a mental illness; a question transmitting module to output the verified set of survey questions through the application; an object determining module to output result data by adjusting the prediction rate of an appearance of symptoms of a mental illness when the set of survey questions is not verified in the branching recommending module; and a result module to transmit the outputted result data; a haptic actuator to provide tactile feedback to guide user engagement, an AI-driven display adjustment module to dynamically modify the interface based on user interaction and cognitive load, a data encryption mechanism to ensure secure storage and transmission of sensitive mental health data, and a dynamic AI model selection process that optimizes model selection in real time based on response patterns and feature importance values. According to an embodiment of the present disclosure, a control signal may be transmitted to a pharmaceutical mixer for a medicine of attention deficit hyperactivity disorder (ADHD) based on the assessment result.

Additionally, the system comprises an EEG sensor to collect and analyze brainwave activity for refining survey questions and assessing cognitive responses.

According to an embodiment of the present disclosure, a method for controlling a pharmaceutical mixer of the ADHD medication based on an assessment of a mental health state of an adolescent (assessed by using a survey formed based on an AI) comprises: an operation of collecting physical information of a user through a pre-installed application, verifying survey questions regarding mental health depending on the physical information of the user, and verifying answers to the verified survey questions inputted by the user through the application; an operation of collecting EEG signal data and using it to refine survey questions; an operation of forming additional survey questions for the user after having verified the answers and calculating a prediction rate of an appearance of symptoms of a mental illness regarding the additional survey questions by using AI models; an operation of verifying a set of survey questions including a plurality of sub-questions depending on the calculated prediction rate of an appearance of symptoms of a mental illness; an operation of dynamically adjusting display elements based on cognitive load and user interaction; an operation of outputting the verified set of survey questions through the application; an operation of outputting result data by adjusting the prediction rate of an appearance of symptoms of a mental illness when the set of survey questions is not verified; and an operation of transmitting the outputted result data; an operation of providing haptic feedback to guide user responses; an operation of encrypting data for secure transmission; an operation of selecting an AI model dynamically based on real-time feature analysis; and an operation of transmitting a control signal to the pharmaceutical mixer of the medication for ADHD based on the assessment result of the selected AI model.

An electronic device for assessing a mental health state of an adolescent by using a survey formed based on an AI and a method therefor have an effect to predict a mental health state of the adolescent and the possibility of an occurrence of a symptom of a mental illness by forming, by means of a machine learning model, survey questions to which the adolescent may easily answer by themselves and analyzing received answers; by integrating additional technical features, such as EEG analysis, haptic feedback, AI-driven display adjustments, encryption-based security, and dynamic AI selection, the accuracy, reliability, and user experience of the assessment process are significantly improved. According to an embodiment of the present disclosure, a control signal may be transmitted to a pharmaceutical mixer for a medicine of attention deficit hyperactivity disorder (ADHD) based on the assessment of a mental health state.

Effects of the present disclosure are not limited to this. Various effects will be described below with reference to respective embodiments.

### BRIEF DESCRIPTION OF THE DRAWINGS

In order that the disclosure may be well understood, there will now be described various forms thereof, given by way of example, reference being made to the accompanying drawings, in which:

FIG. 1 is a block diagram illustrating an AI-based mental health assessment system incorporating a survey system based on AI, haptic actuator module, AI-driven display adjustments module, data encryption and secure transmission module, and dynamic AI model selection process module according to an embodiment of the present disclosure;

FIG. 2 is a block diagram illustrating a configuration of a question collecting module according to an embodiment of the present disclosure;

FIG. 3 is a block diagram illustrating a configuration of an AI model module according to an embodiment of the present disclosure;

FIG. 4 is a block diagram illustrating a configuration of a branching recommending module according to an embodiment of the present disclosure;

FIG. 5 is a block diagram illustrating a configuration of an object determining module according to an embodiment of the present disclosure;

FIG. 6 is a block diagram illustrating a configuration of a result module according to an embodiment of the present disclosure;

FIG. 7 is a block diagram illustrating a configuration of a haptic actuator module according to an embodiment of the present disclosure;

FIG. 8 is a block diagram illustrating a configuration of an AI-driven display adjustments module according to an embodiment of the present disclosure;

FIG. 9 is a block diagram illustrating a configuration of a data encryption and secure transmission module according to an embodiment of the present disclosure;

FIG. 10 is a block diagram illustrating a configuration of a dynamic AI model selection process module;

FIG. 11 is a block diagram illustrating a configuration of an EEG sensor integration and processing module according to an embodiment of the present disclosure;

FIG. 12 is a diagram showing a data set formed by collecting physical information of a user and available for training according to an embodiment of the present disclosure;

FIG. 13 is a diagram showing a result of a survey in an early stage made as data according to an embodiment of the present disclosure;

FIG. 14 is a diagram showing optimum survey questions formed by using AI models according to an embodiment of the present disclosure;

FIG. 15 is a graph showing results of trainings for performance of AI models by using data of entire questions according to an embodiment of the present disclosure;

FIG. 16 is a graph showing performance of AI models re-trained by using data of optimum survey questions formed depending on AI models according to an embodiment of the present disclosure;

FIGS. 17A and 17B are a diagram showing a decision tree model in which prediction values are branched into multiple AI models according to an embodiment of the present disclosure;

FIG. 18 is a graph showing results of verification of performance of trained AI models according to an embodiment of the present disclosure; and

FIG. 19 is a graph for determining an object for a periodic management by using prediction values of a plurality of AI models according to an embodiment of the present disclosure.

FIG. 20 is a block diagram illustrating that a control signal is transmitted to a pharmaceutical mixer of a ADHD medication based on the assessment result according to an embodiment of the present disclosure.

FIG. 21 is a flow chart illustrating a method for controlling a pharmaceutical mixer of the ADHD medication based on an assessment of a mental health state of an adolescent by using a bioelectrical activity data collected by an electroencephalogram (EEG) sensor along within responses to a survey formed by using AI models.

FIG. 22 is a flow chart illustrating supplemental methods how the AI models are utilized for the assessment of a mental health state.

### DETAILED DESCRIPTION OF EXEMPLARY EMBODIMENTS

This specification may use the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” may refer to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, an Apache MXNet framework, or etc.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Since a technology described below may have various modifications and various embodiments, specific embodiments will be illustrated in the accompanying figures and described in detail. However, it should be understood that the below descriptions include all modifications, equivalents or substitutes which belong to the ideas and the scope of the described technology.

Terms, such as ‘first’, ‘second’, ‘A’, ‘B’ or the like may be used to describe various components. However, these terms do not limit the components, but are used only for distinguishing one component from another. For example, without being out of the scope of the right of the below-described technology, a first component may be referred to as a second component and, in a similar way, a second component may be referred to as a first component. The term ‘and/or’ means a combination of the relevant descriptions or any one of the relevant descriptions. For example, the description ‘A and/or B’ may be interpreted as ‘at least one of A and B’. In addition, the mark ‘/’ may be interpreted as ‘and’ or ‘or’.

In this specification, a term in a singular form may also mean a term in a plural form as long as there is no particular indication. It should be understood that the term ‘comprise’, etc. means the existence of the described characteristics, numbers, steps, operations, components, parts or their combinations, but does not mean the exclusion of the existence or the possibility of addition of one or more other characteristics, numbers, steps, operations, components, parts or their combinations.

Before describing the drawings in detail, it should be clarified that the classification of the components in this specification is made only by their main functions. That is, it is possible that two or more below-described components may be combined as one component or one component may be divided into two or more components depending on their detailed functions. In addition, each of the below-described components may additionally perform, in addition to its own main functions, some or all functions that another component performs. Otherwise, some of the main functions of each component may also be exclusively performed by another component.

In a method or an operational method, as long as any specific order is clearly indicated, steps for the method may be performed in an order different from the order in which the steps are described. That is, the steps may be performed in the order in which they are described, simultaneously, or in a reverse order.

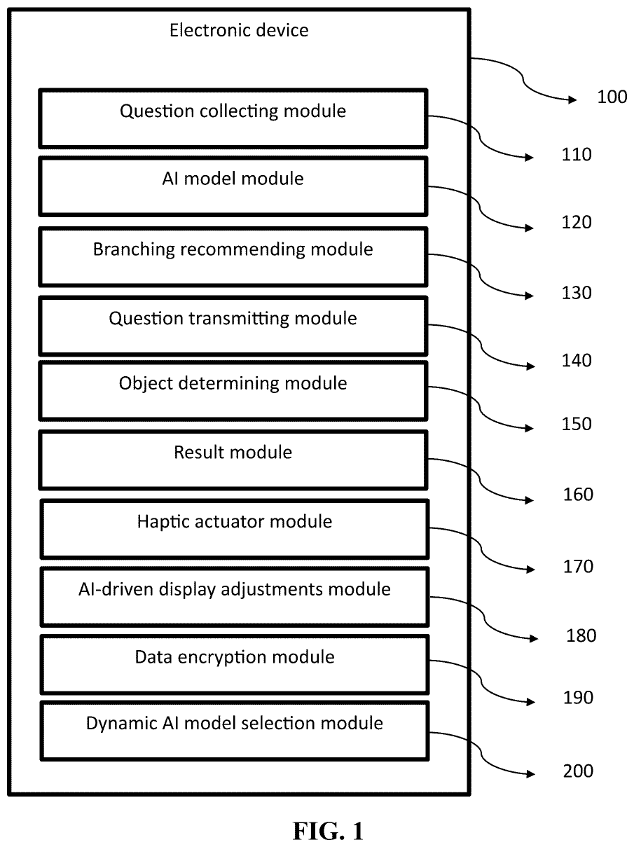

FIG. 1 is a block diagram illustrating a configuration of an electronic device for assessing a mental health state of a user by using a survey based, in combination with real-time neurophysiological data obtained via an EEG sensor, on an AI according to an embodiment of the present disclosure. As described above, the various modules of exemplary embodiments (described hereafter) can be implemented as one or more computer programs, i.e., one or more modules of computer program instructions encoded on a tangible non transitory storage medium for execution by, or to control the operation of, data processing apparatus.

Referring to FIG. 1, an electronic device 100 may comprise a question collecting module 110, an AI model module 120, a branching recommending module 130, a question transmitting module 140, an object determining module 150, a result module 160, a haptic actuator module 170, an AI-driven display adjustments module 180, a data encryption module 190, and a dynamic AI model selection module 200. In addition to them, other various components for collecting items for a survey based, in combination with real-time neurophysiological data obtained via an EEG sensor 300, on an AI and assessing a mental health state based on answers of a user may be comprised.

According to an embodiment of the present disclosure, the electronic device 100 may assess a mental health state of a user and predict the possibility of an appearance of symptoms of a mental illness when the user inputs answers to questions for a survey by using an application comprising a technology of the subject disclosure. In addition, the electronic device 100 may transmit the answers of the user to an external server so that the server may perform an assessment of a mental health state of the user and a prediction of the possibility of an appearance of symptoms of a mental illness of the user.

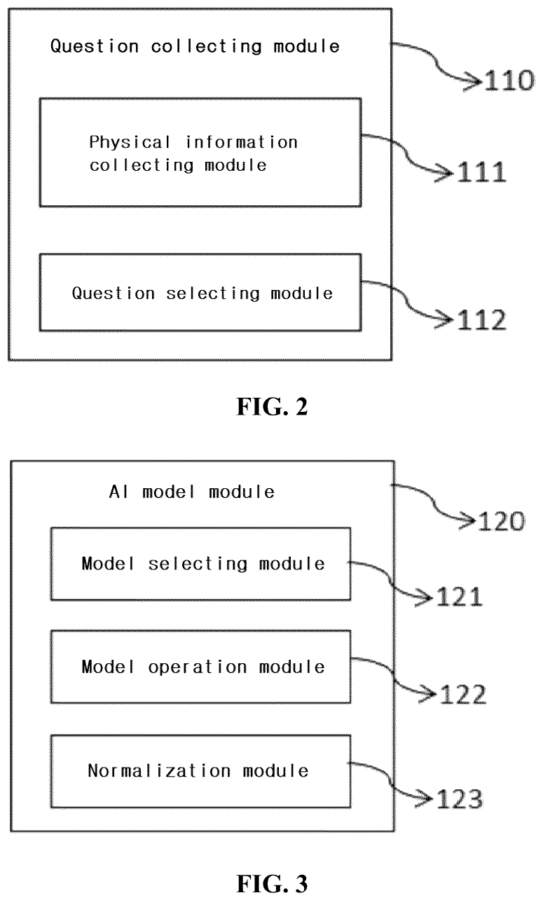

According to an embodiment of the present disclosure, the question collecting module may control optimum questions for the survey based on physical information of the user collected by the application to be automatically presented to support the user's answers to the questions of the survey. A more specific description of the question collecting module 110 will be made below with reference to FIG. 2.

According to an embodiment of the present disclosure, the AI model module 120 may perform an operation allowing the prediction by forming data for inference regarding an optimum survey or additional questions, given to a user by a sub-question assigning module, to which answers are completed and being branched into a plurality of trained AI models to distinguish a symptom group from a normal group. More specific description of the AI model module 120 will be made below with reference to FIG. 3.

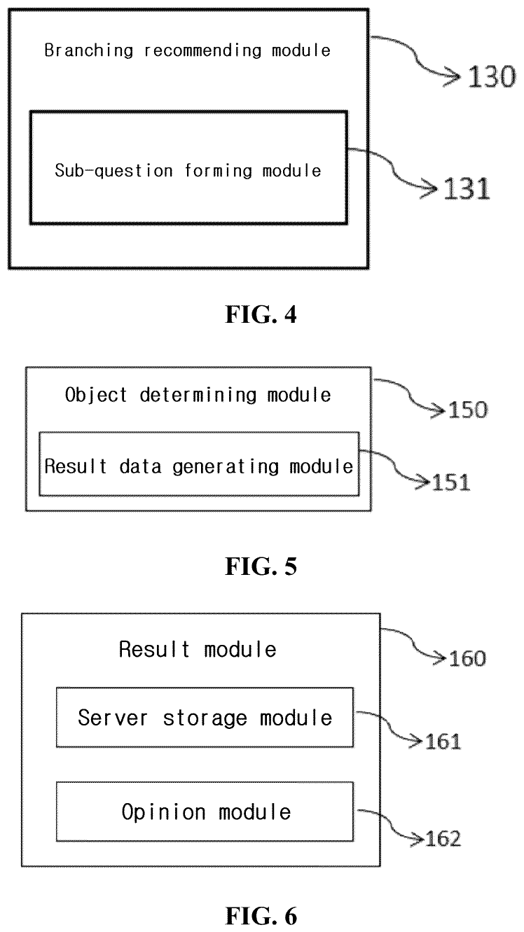

According to an embodiment of the present disclosure, when a prediction rate of a symptom appearance is equal to or higher than a reference value based on a prediction probability calculated in the AI model module 120, the branching recommending module may search for sets of survey questions comprising more sub-questions for enhancing reliability of a model. More specific description of the branching recommending module will be made below with reference to FIG. 4.

According to an embodiment of the present disclosure, the question transmitting module may transmit sets of additional survey questions outputted by the branching recommending module 130 to the user through the application. For example, the question transmitting module 140 may provide the latest information to the user by using additional questions outputted by the branching recommending module 130.

According to an embodiment of the present disclosure, the question transmitting module may secure reliability of additional questions by deriving from the user determinations easily influenced by the latest information and transmit notifications through the application so that the user may periodically use the application. As such, the question transmitting module 140 may enhance the prediction rate of the AI model by using answers to the additional questions presented to the user.

According to an embodiment of the present disclosure, the object determining module may determine an adjusted prediction value based a threshold value indicating that there is no further model to be branched and output result data based on the number of branches of models. More specific description of the object determining module 150 will be made below with reference to FIG. 5.

According to an embodiment of the present disclosure, the result module 160 may transmit to a psychiatrist a comprehensive result including the answers to the survey questions, the number of branches of models, classifications of the survey, or the like by using result data produced by the object determining module 150. More specific description of the result module 160 will be made below with reference to FIG. 6.

According to an embodiment of the present disclosure, the haptic actuator module 170 may provide sensory feedback to the user by analyzing responses and engagement levels during the survey process, thereby enhancing user interaction and engagement with the mental health assessment system. The haptic actuator module 170 may generate adaptive feedback signals based on AI-analyzed mental state predictions to guide user responses and reduce stress. More specific description of the haptic actuator module 170 will be made below with reference to FIG. 7.

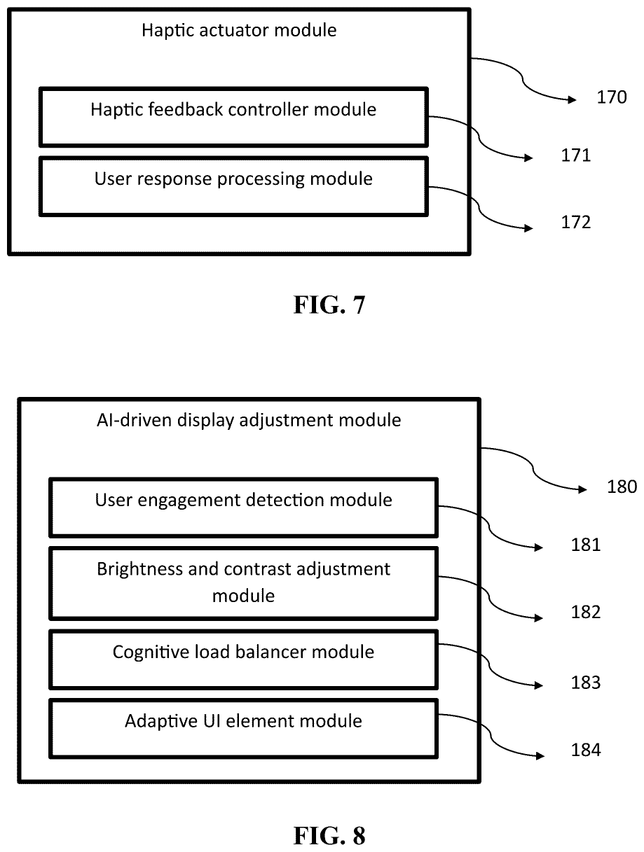

According to an embodiment of the present disclosure, the AI-driven display adjustments module 180 may optimize the visual presentation of the survey interface by dynamically modifying screen brightness, contrast, and UI (user interface) elements based on the cognitive load and engagement levels of the user. The AI-driven display adjustment module 180 may ensure that the mental health assessment remains accessible and minimizes user fatigue. More specific description of the AI-driven display adjustment module 180 will be made below with reference to FIG. 8.

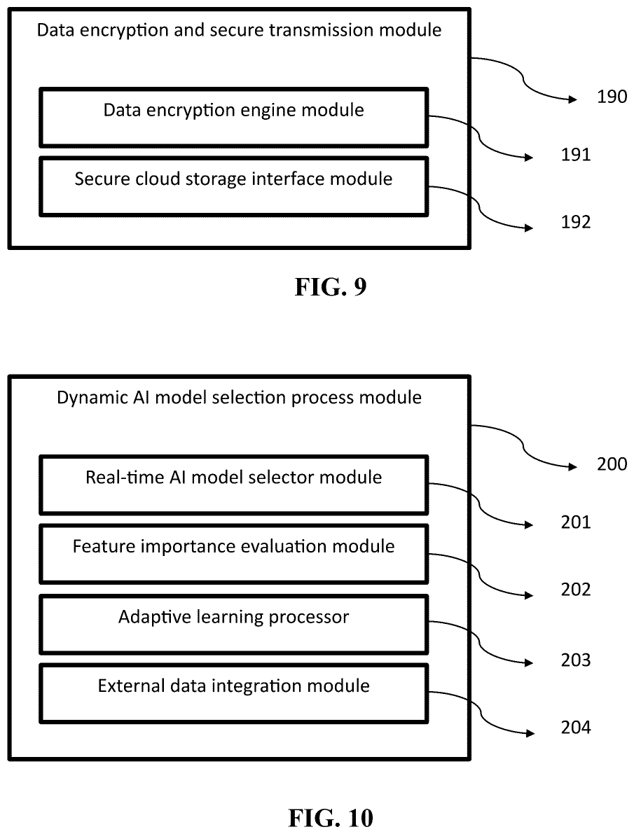

According to an embodiment of the present disclosure, the data encryption module 190 may ensure secure processing and transmission of sensitive mental health data collected during the survey and EEG measurements. The data encryption module 190 may employ encryption protocols to protect data integrity and confidentiality while enabling secure cloud storage and retrieval by authorized psychiatrists. More specific description of the data encryption module 190 will be made below with reference to FIG. 9.

According to an embodiment of the present disclosure, the dynamic AI model selection module 200 may enhance the predictive accuracy of the mental health assessment system by selecting and adapting AI models in real time based on user response patterns and external data inputs. The dynamic AI model selection module 200 may dynamically integrate multiple AI models to refine the prediction of mental health symptoms, ensuring adaptability to diverse user profiles. More specific description of the dynamic AI model selection module 200 will be made below with reference to FIG. 10.

FIG. 2 is a block diagram illustrating a configuration of a question collecting module according to an embodiment of the present disclosure.

Referring to FIG. 2, the question collecting module 110 may comprise a physical information collecting module 111 and a question selecting module 112.

According to an embodiment of the present disclosure, the question collecting module may induce a user to quickly answer a survey by collecting physical information of the user through an application, to which an AI model is applied, and presenting to the user optimum survey questions classified by using collected physical information of the user. In addition, the question collecting module 110 may generate a data set for the user comprising the optimum questions for deriving a primary effect.

According to an embodiment of the present disclosure, the physical information collecting module 111 may ask various questions regarding physical information of the user, such as age, height, weight, waist measurement, etc. and receive answers from the user. For example, the physical information collecting module 111 may collect physical information of the user based on the answers received from the user.

According to an embodiment of the present disclosure, the question selecting module 112 may present optimum survey questions selected by the AI model based on the physical information of the user collected by the physical information collecting module 111. For example, the question selecting module 112 may present optimum survey questions based on the physical information of the user so as to obtain reliable answers within a short time.

In a general survey in which answers to all the questions are required, the more questions there are, the more time it takes for a user to answer. In addition, the reliability of the user's answers may also decrease. Further, in a case when it is the first time for a user to use an application for a survey, it is highly likely that the brain of the user negatively perceives the application. On the other hand, according to the present disclosure, since the question selecting module 112 presents to a user optimum survey questions based on physical information of the user, the user can answer a relatively small number of questions and questions based on the physical information of the user's own. This leads to a reduced time for the answers and an enhanced reliability of the answers.

FIG. 3 is a block diagram illustrating a configuration of an AI model module according to an embodiment of the present disclosure.

Referring to FIG. 3, the AI model module 120 may comprise a model selecting module 121, a model operation module 122, and a normalization module 123.

According to an embodiment of the present disclosure, the AI model module 120 may predict an appearance of symptoms of a mental illness of the user by using a user data set generated by the question collecting module 110.

According to an embodiment of the present disclosure, the model selecting module 121 may select an AI model for prediction depending on the number of questions used for the survey. For example, the AI model may be a machine learning model such as Logistic Regression, Decision Tree, Random Forest, XGBoost, LightGBM or the like.

According to an embodiment of the present disclosure, the model operation module 122 may calculate a prediction value of an AI model selected by the model selecting module 121. For example, the model operation module 122 may control a plurality of AI models to perform predictions about the questions answered by the user and to perform final predictions by using weightings for prediction values set for respective AI models. In addition, the model operation module 122 may multiply the weightings set for the respective AI models and the prediction values to obtain adjusted prediction values and operate final prediction values by comparing these values.

According to an embodiment of the present disclosure, the model operation module 122 may generate a new feature by means of equation 1 using prediction values of multiple AI models and entropies of leaf nodes. For example, the model operation module 122 may generate a new feature by calculating adjusted prediction values of the respective models through the multiplication of the prediction values and the entropies or the weightings and adding up the calculated values.

Equation 1 is to generate a new feature by using the prediction values of the multiple AI models and the entropies of the leaf nodes according to an embodiment of the present disclosure.

$${F}_{n e w} = {\sum }_{i = 1}^{N} ( {P}_{i} ) \qquad [ Equation 1 ]$$

$${P}_{i} = {p}_{i} {W}_{i}$$

According to an embodiment of the present disclosure, F_{new} is a new feature generated by equation 1. N is a total number of a plurality of AI models presented in the present disclosure. Pᵢ is an adjusted prediction value of each AI model, which is based on a prediction value and a weighting, and pᵢ is a prediction value of each AI model. Wᵢ means an entropy in a decision tree model and a weighting in other models.

According to an embodiment of the present disclosure, the normalization module 123 may verify the AI models in their prediction reliabilities based on final prediction values outputted from the model operation module 122 by using 7 performance evaluation indexes: receiver operating characteristics curve, accuracy, sensitivity, specificity, precision, recall, and F1 score. In addition, the normalization module 123 may also indicate evaluation indexes of a psychiatrist for prediction models in numbers.

According to an embodiment of the present disclosure, the evaluation indexes of a psychiatrist may be levels of prediction accuracy of AI models represented by numbers by a psychiatrist evaluating symptom predictions of AI models based on data used for trainings through the comparison with symptoms and diagnoses data obtained by using a screening tool such as the Kiddle Schedule for Affective Disorders and Schizophrenia Present and Lifetime Version (K-SADS-PL).

FIG. 4 is a block diagram illustrating a configuration of a branching recommending module according to an embodiment of the present disclosure.

Referring to FIG. 4, the branching recommending module 130 may comprise a sub-question forming module 131.

According to an embodiment of the present disclosure, the branching recommending module 130 may determine whether sub-questions need to be formed based on prediction values of the possibility of an appearance of symptoms of a mental illness outputted from the AI model module 120 and generate a new set of survey questions by selecting additional questions. For example, the branching recommending module 130 may construct, based on classification of survey questions, an AI model trained by entire questions and a plurality of AI models trained by a reduced number of survey questions including only questions important in respective groups.

According to an embodiment of the present disclosure, the sub-question forming module may generate additional questions by using the constructed AI models and collect only answers to the additional questions as new data by comparing the generated additional questions with the questions to which the user has already answered.

Equation 2 is for a calculation reflecting feature importance values in a plurality of AI models.

$$SI = \{ {IMP}_{f 1} , {IMP}_{f 2} , … , {IMP}_{fN} \} \qquad [ Equation 2 ]$$

$${IMP}_{fN} = \vert \frac{( {IMP}_{Af 1} + {IMP}_{Bf 1} + {IMP}_{Cf 1} + … + {IMP}_{Mf 1} )}{{N}_{models}} \vert$$

According to an embodiment of the present disclosure, SI is a set of collected feature importance values. IMP is an importance value. N is a number of a feature. A-M are identification information of AI models A to M. IMP_{fN} is a final importance value of one feature derived from a plurality of AI models. IMP_{Mf1} is an importance value of a first feature of a model. N_{models} is the number of AI models.

According to an embodiment of the present disclosure, an importance value of a first feature of each AI model may be represented in a form of IMP_{Mf1} and the branching recommending module 130 may calculate an average importance value for the first features by adding up the importance values for the first features of all the models and dividing the sum by the number of the models. The branching recommending module may form a set by repeating the operation for calculating an average importance value as many times as the number of the entire features.

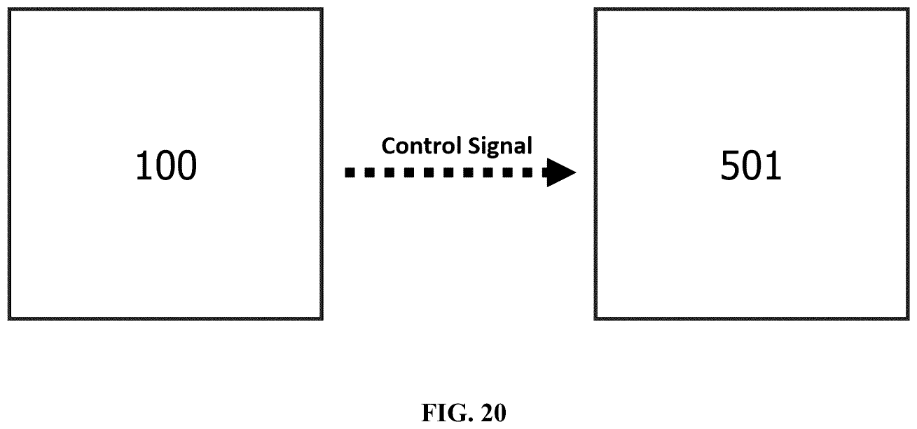

According to an embodiment of the present disclosure, the branching recommending module 130 may output additional questions, generated based on equation 2, to a user in an order of importance values. For example, the electronic device 100 may comprise an output device (not shown) for outputting additional questions in various forms of data (for example, sound, image, etc.) and the branching recommending module 130 may output additional questions through the output device. Further, according to an embodiment, (as shown in FIG. 20) a control signal may be transmitted to a pharmaceutical mixer 501 of a medication for Attention Deficit Hyperactivity Disorder (ADHD) based on the assessment result.

FIG. 5 is a block diagram illustrating a configuration of an object determining module according to an embodiment of the present disclosure.

Referring to FIG. 5, the object determining module 150 may comprise a result data generating module 151.

According to an embodiment of the present disclosure, the object determining module may determine, based on a prediction result outputted from the branching recommending module 130, a user as an object for management by annual periods or monthly periods. For example, the object determining module 150 may verify the possibility of an appearance of symptoms of a mental illness by using prediction values after the formation of the additional questions and determine a user, having a prediction value higher than a predetermined threshold value, as an object for management by periods.

According to an embodiment of the present disclosure, the result data generating module may output result data to support the determination of a psychiatrist by providing answers to survey questions used by including a variable of an object for periodic management and prediction values of a plurality of models.

FIG. 6 is a block diagram illustrating a configuration of a result module according to an embodiment of the present disclosure.

Referring to FIG. 6, the result module 160 may comprise a server storage module 161 and an opinion module 162.

According to an embodiment of the present disclosure, the result module 160 may store result data outputted from the object determining module 150 in a database and transmit an opinion of a psychiatrist as data in a form for an application or a web service to a user's electronic device.

According to an embodiment of the present disclosure, the server storage module 161 may store result data in a database server in a form of a cloud or on-premise solution in the manner of de-identification.

According to an embodiment of the present disclosure, when the result data has completed to be stored, the opinion module 162 may generate data in a form for an application or a web service with the stored result data so that a psychiatrist may refer thereto. Then, the psychiatrist may transfer their opinion in a form of data for an application or a web service to a user.

FIG. 7 is a block diagram illustrating a configuration of a haptic actuator module according to an embodiment of the present disclosure.

Referring to FIG. 7, the haptic actuator module 170 may comprise a haptic feedback controller module 171 and a user response processing module 172.

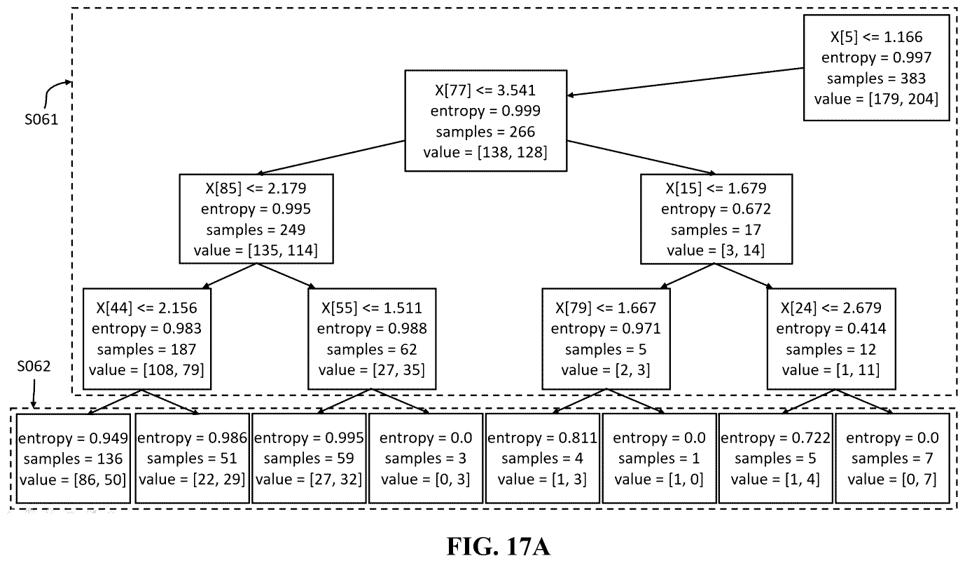

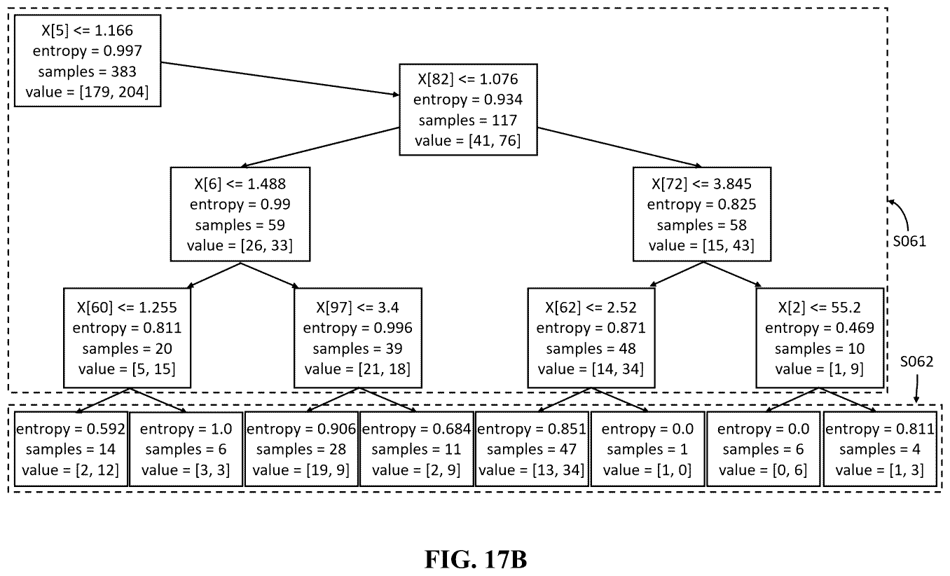

According to an embodiment of the present disclosure, the haptic actuator module 170 may provide sensory feedback to the user based on real-time analysis of survey responses, EEG data, and AI-driven mental health assessment outcomes. The haptic actuator module may receive data from the AI model module 120, the EEG sensor integration and processing module 300, and the question collecting module 110 to dynamically generate feedback signals that improve user engagement and response accuracy. The generated haptic feedback may be tailored to the user's emotional and cognitive state, helping regulate stress levels and improving the survey-taking experience. More specific descriptions of the haptic actuator module 170 will be made below with reference to FIG. 17.

According to an embodiment of the present disclosure, the haptic feedback controller module 171 may regulate the strength, duration, and pattern of haptic responses based on the user's survey responses and EEG-derived neurophysiological data. The module may process input from the user response processing module 172 to determine the appropriate level of feedback, such as gentle vibrations for stress relief or rhythmic pulses to maintain focus.

According to an embodiment of the present disclosure, the user response processing module 172 may analyze user input patterns, including touch gestures, response latency, and consistency in survey answers, to refine haptic feedback delivery. The module may communicate with the question collecting module 110 to assess changes in user engagement and modify haptic interactions accordingly. For example, if a user exhibits hesitation or erratic answering behavior, the module may trigger calming haptic pulses to encourage a steady response flow.

FIG. 8 is a block diagram illustrating a configuration of an AI-driven display adjustments module according to an embodiment of the present disclosure.

Referring to FIG. 8, the AI-driven display adjustments module 180 may comprise a user engagement detection module 181, a brightness and contrast adjustment module 182, a cognitive load balancer module 183, and an adaptive UI element module 184.

According to an embodiment of the present disclosure, the AI-driven display adjustments module 180 may optimize the user interface in real time based on cognitive load, emotional state, and user engagement levels. The module may receive data from the EEG sensor integration and processing module 300, the haptic actuator module 170, and the AI model module 120 to dynamically adjust display elements, including brightness, contrast, and content layout. The AI-driven display adjustments module 180 may improve user focus and enhance the accessibility of survey content, ensuring that users remain engaged without experiencing visual fatigue. More specific descriptions of the AI-driven display adjustments module 180 will be made below with reference to FIG. 8.

According to an embodiment of the present disclosure, the user engagement detection module 181 may monitor user interactions, such as eye-tracking data, response delays, and screen activity, to determine engagement levels. The module may adjust display elements based on detected fluctuations in attention and work in conjunction with the cognitive load balancer module 183 to optimize information delivery. For instance, if engagement levels drop, the module may trigger subtle visual cues to re-capture user focus.

According to an embodiment of the present disclosure, the brightness and contrast adjustment module 182 may modify screen settings dynamically to enhance readability and reduce visual strain. The module may analyze ambient lighting conditions, user preferences, and EEG-derived indicators of cognitive load to optimize screen brightness and contrast. Additionally, it may adjust text size and background colors to accommodate different levels of user alertness or fatigue.

According to an embodiment of the present disclosure, the cognitive load balancer module 183 may regulate the complexity of displayed content based on real-time user data. The module may work alongside the user engagement detection module 181 and the EEG sensor integration and processing module 300 to assess cognitive burden and adapt survey presentation accordingly. For example, if a user exhibits signs of cognitive overload, the module may simplify displayed text or break down complex questions into smaller components.

According to an embodiment of the present disclosure, the adaptive UI element module may dynamically rearrange interface elements to enhance user experience and improve response accuracy. The module may personalize button placements, adjust scroll speeds, and introduce animation effects to guide user focus toward critical information. In coordination with the haptic actuator module 170, the adaptive UI element module 184 may also synchronize visual adjustments with haptic feedback for a multimodal user experience.

FIG. 9 is a block diagram illustrating a configuration of a data encryption and secure transmission module according to an embodiment of the present disclosure.

Referring to FIG. 9, the data encryption and secure transmission module 190 may comprise a data encryption engine module 191 and a secure cloud storage interface module 192.

According to an embodiment of the present disclosure, the data encryption and secure transmission module 190 may ensure the protection and confidentiality of user data collected during mental health assessments. This module may encrypt sensitive user information, including EEG sensor readings and survey responses, before transmitting it to external servers for further analysis. The data encryption and secure transmission module 190 may work in coordination with the AI model module 120, the EEG sensor integration and processing module 300, and the server storage module 161 to prevent unauthorized access and ensure secure storage and retrieval. More specific descriptions of the data encryption and secure transmission module 190 will be made below with reference to FIG. 9.

According to an embodiment of the present disclosure, the data encryption engine module may apply industry-standard encryption algorithms such as Advanced Encryption Standard (AES) and Rivest-Shamir-Adleman (RSA) cryptographic techniques to protect user data. The module may generate unique encryption keys for each session, ensuring that sensitive information remains secure throughout data processing and transmission.

According to an embodiment of the present disclosure, the secure cloud storage interface module 192 may facilitate the transmission of encrypted data to cloud-based servers while maintaining compliance with data protection regulations. This module may employ end-to-end encryption protocols to protect data integrity and ensure that user information remains accessible only to authorized entities, such as psychiatrists or healthcare providers.

FIG. 10 is a block diagram illustrating a configuration of a dynamic AI model selection process module.

Referring to FIG. 10, the dynamic AI model selection module 200 may comprise a real-time AI model selector module 201, a feature importance evaluation module 202, an adaptive learning processor 203, and an external data integration module 204.

According to an embodiment of the present disclosure, the dynamic AI model selection module 200 may optimize mental health assessment by dynamically selecting the most suitable AI model based on user data characteristics, EEG readings, and survey responses. This module may ensure that the AI model used for prediction and analysis is continuously updated and adjusted to provide the most accurate mental health assessment. The dynamic AI model selection module 200 may work in conjunction with the AI model module 120, the EEG sensor integration and processing module 300, and the data encryption and secure transmission module 190 to enhance adaptability and predictive accuracy. More specific descriptions of the dynamic AI model selection module 200 will be made below with reference to FIG. 10.

According to an embodiment of the present disclosure, the real-time AI model selector module 201 may evaluate multiple AI models and select the one most suited to the current dataset. The selection criteria may include prediction accuracy, processing efficiency, and adaptability to new survey or EEG data. The module may dynamically update the AI model in real-time as new data is collected to improve accuracy and personalization.

According to an embodiment of the present disclosure, the feature importance evaluation module 202 may analyze user responses and EEG data to determine which features contribute most significantly to mental health assessments. This module may rank and weigh input variables, ensuring that the AI model selection process prioritizes the most relevant indicators for accurate predictions.

According to an embodiment of the present disclosure, the adaptive learning processor may enable AI models to learn from user responses and EEG data over time. The module may continuously refine model parameters and optimize prediction performance based on longitudinal data patterns, allowing for improved mental health monitoring and adaptation to individual users.

According to an embodiment of the present disclosure, the external data integration module 204 may incorporate external datasets, such as clinical research data or psychiatric diagnostic databases, into the AI model selection process. This module may allow the dynamic AI model selection module 200 to enhance its predictive capabilities by leveraging broader datasets while ensuring compliance with data security and privacy standards.

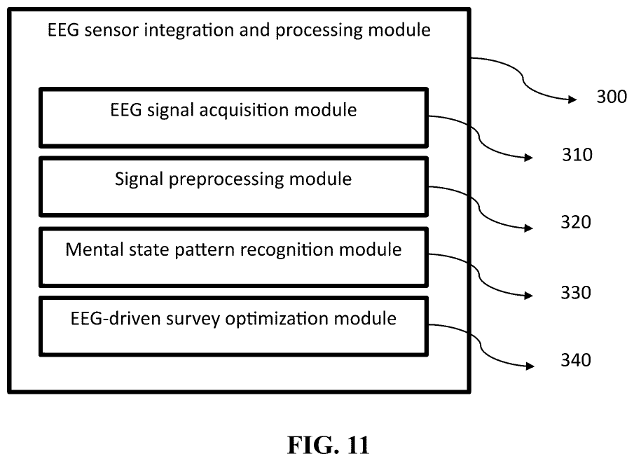

FIG. 11 is a block diagram illustrating a configuration of an EEG sensor integration and processing module according to an embodiment of the present disclosure.

Referring to FIG. 11, an EEG sensor module 300 may comprise an EEG signal acquisition module 310, a signal preprocessing module 320, a mental state pattern recognition module 330, and an EEG-driven survey optimization module 340. In addition to them, other various components for collecting neurophysiological data in real time, in combination with AI-driven analysis and survey responses, may be used to assess a mental health state based on both bio-signals and user input.

According to an embodiment of the present disclosure, the EEG sensor integration and processing module 300 may facilitate the real-time collection, processing, and interpretation of neurophysiological data to enhance mental health assessment. This module may work in conjunction with the AI model module 120, the question collecting module 110, and the dynamic AI model selection module 200 to improve the precision and adaptability of mental health evaluations by incorporating objective biological data. The EEG sensor integration and processing module 300 may continuously monitor brainwave activity to provide supplementary data for AI-driven predictions. More specific descriptions of the EEG sensor integration and processing module 300 will be made below with reference to FIG. 11.

According to an embodiment of the present disclosure, the EEG signal acquisition module 310 may collect raw electrical signals from the brain using EEG electrodes placed on the user's scalp or a wearable EEG device. The module may digitize and transmit these signals to the processing units while filtering out noise and external interferences.

According to an embodiment of the present disclosure, the signal preprocessing module may refine EEG data by removing artifacts such as eye blinks, muscle movements, and external electronic interferences. This module may apply filtering techniques, such as Fast Fourier Transform (FFT) or Independent Component Analysis (ICA), to extract relevant neural patterns for further analysis.

According to an embodiment of the present disclosure, the mental state pattern recognition module 330 may analyze preprocessed EEG signals to detect cognitive and emotional states relevant to mental health assessments. This module may classify brainwave activity into different frequency bands (e.g., alpha, beta, theta) and correlate specific patterns with emotional states, cognitive load, or stress indicators.

According to an embodiment of the present disclosure, the EEG-driven survey optimization module 340 may dynamically adjust survey questions based on real-time EEG data. If the module detects elevated stress levels or cognitive fatigue, it may modify the difficulty, structure, or pacing of the survey to ensure an optimal user experience and more reliable response data.

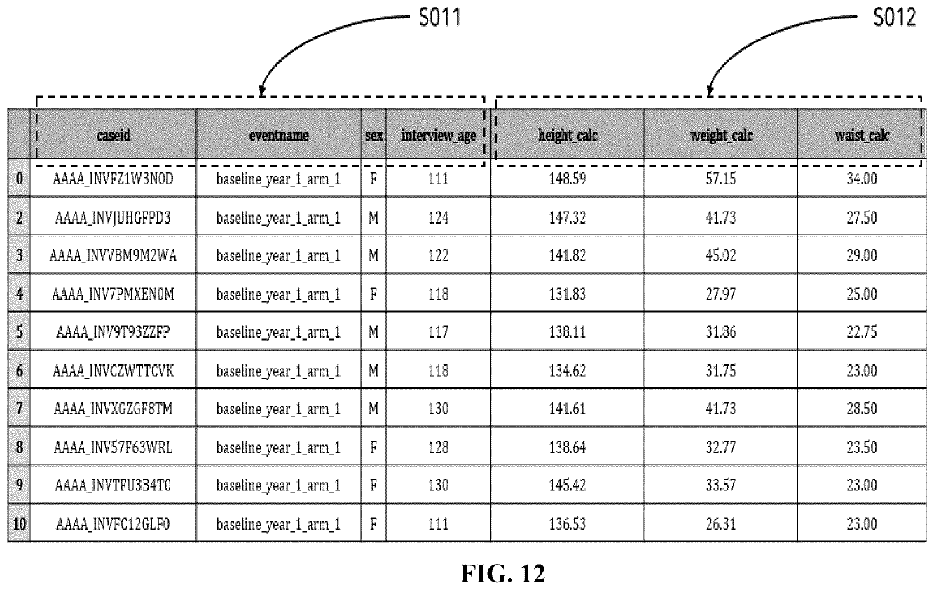

FIG. 12 is a diagram showing a data set formed by collecting physical information of a user and available for training according to an embodiment of the present disclosure.

Referring to FIG. 12, the question collecting module 110 may collect physical information of a user by using the physical information collecting module 111 and verify survey questions based on the collected physical information by using the question selecting module 112.

According to an embodiment of the present disclosure, the question collecting module may verify and store, by unique identifiers (caseid) of users used inside a system, collected physical information such as times of a survey of users (event name), the sexes of users (sex), the monthly ages of users (interview_age) as indicated by S011.

According to an embodiment of the present disclosure, the question collecting module may verify and store, as physical information for the unique identifiers, heights of users (height_calc), weights of users (weight_calc), and waist measurements of users (waist_calc) as indicated by S012.

According to an embodiment of the present disclosure, the question collecting module may perform the above described operations indicated by S011 and S012 based on information inputted by users through an application or a web page.

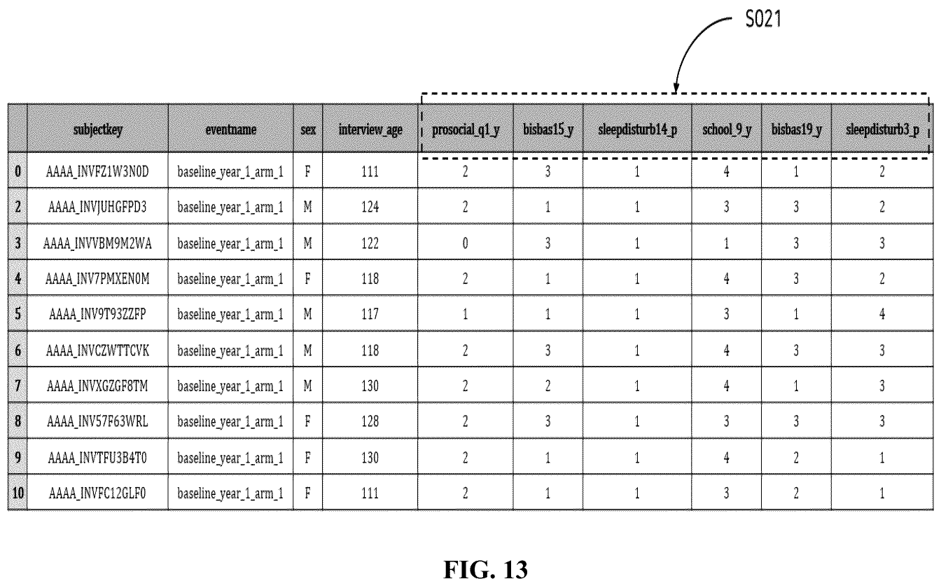

FIG. 13 is a diagram showing a result of a survey in an early stage made as data according to an embodiment of the present disclosure.

Referring to FIG. 13, the question collecting module 110 may verify optimum survey questions selected by AI models at an early stage of a service use and transmit them to a user.

According to an embodiment of the present disclosure, the question collecting module may verify and store scores regarding respective questions, which are a question regarding pro-sociality (prosocial), a question regarding a behavior inhibition system (bisbas), a question regarding a behavioral activation system, a question regarding a sleep disturbance scale (sleep disturb), and a question regarding a school life (school) as indicated by S021, based on the physical information of the user.

According to an embodiment of the present disclosure, the question collecting module may verify values inputted in items of a time of a survey of a user (eventname), the sex of a user (sex), and the monthly age of a user (interview_age) and assign values for answer data regarding respective questions based on score sections predetermined to correspond to the respective input values.

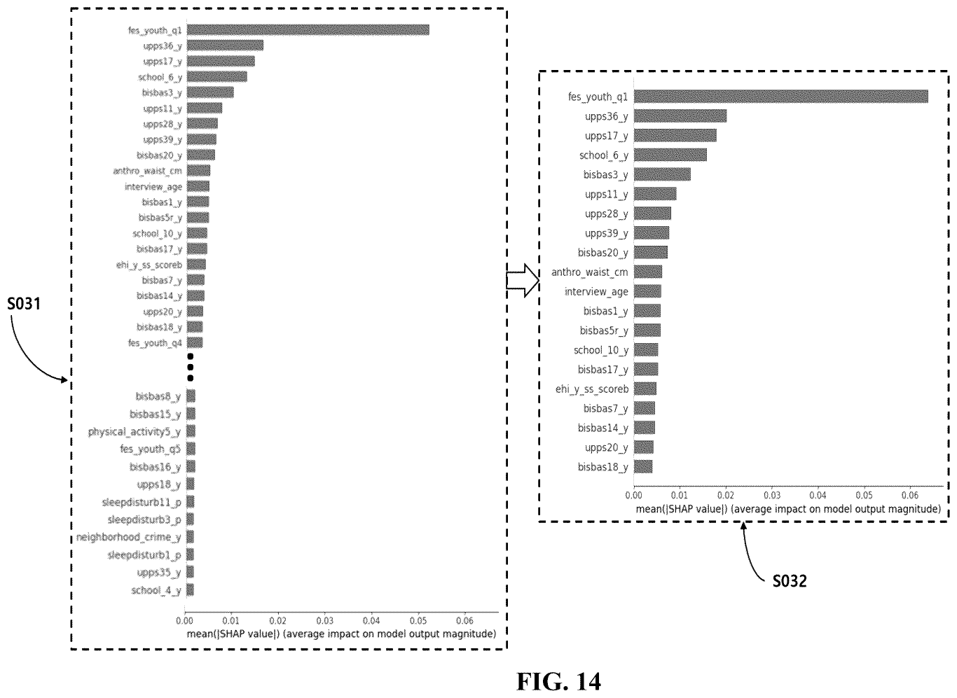

FIG. 14 is a diagram showing optimum survey questions formed by using AI models according to an embodiment of the present disclosure.

Referring to FIG. 14, the question collecting module 110 may verify questions to be important features for prediction by using a plurality of AI models, arrange the questions in a descending order based on importance values of the respective verified questions, and verify features of high-ranked questions within a reference range.

According to an embodiment of the present disclosure, the question collecting module may determine importance of each of all the features based on a plurality of AI models as shown in S031. For example, the question collecting module 110 may determine high-ranked features within a range, in which the performance decrease of an AI model is minimized, based on the importance of each of all the features determined above. Also, according to an embodiment, (as shown in FIG. 20) a control signal can be transmitted to a pharmaceutical mixer 501 of a medication for Attention Deficit Hyperactivity Disorder (ADHD) based on the assessment result.

According to an embodiment of the present disclosure, the question collecting module may form survey questions including the high-ranked features determined above as shown in S032 and transmit them to a user. For example, the question collecting module may form a number of survey questions, in which the number is predetermined for a corresponding user, and an optimum number may be set based on information related to a user.

According to an embodiment of the present disclosure, since the question collecting module 110 forms survey questions including high-ranked features, the number of survey questions may be reduced from 104 in total to 20.

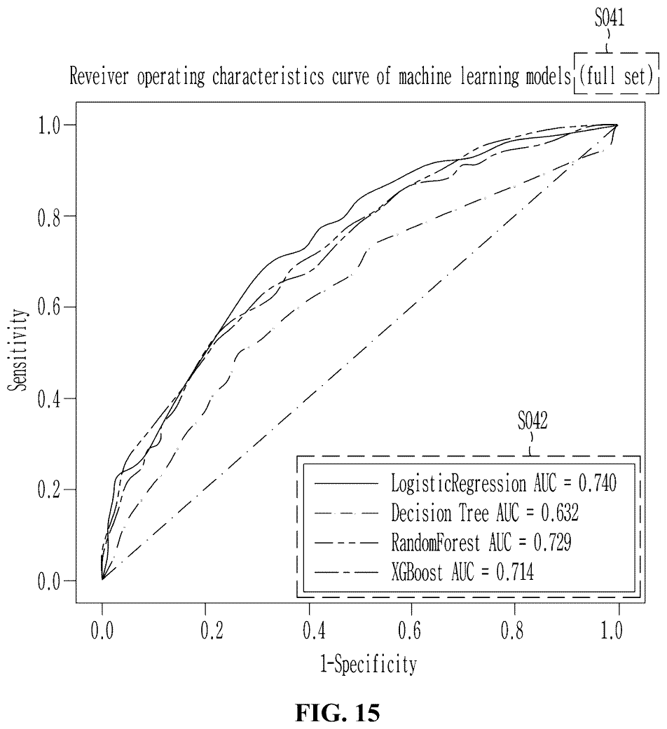

FIG. 15 is a graph showing results of trainings for performance of AI models by using data of entire questions according to an embodiment of the present disclosure.

Referring to FIG. 15, the AI model module 120 may train AI models with collected data of users, verify the data, separate a test set of data from the verified data, and test performance of the AI models with the test set. For example, the AI model module 120 may evaluate the performance of the AI models with evaluation indexes of specificity and sensibility and verify evaluation values for the respective evaluation indexes.

According to an embodiment of the present disclosure, the AI model module 120 may output entire survey questions by using a plurality of AI models S042 and show the performances of the AI models regarding the outputted entire survey questions represented by a graph S041 with evaluation values for the specificity and the sensibility.

According to an embodiment of the present disclosure, although 4 types of AI models as a plurality of AI models S042 are represented in the figure, the AI model module 120 may set the number of AI models to be used depending on physical information of users or the numbers of survey questions for users.

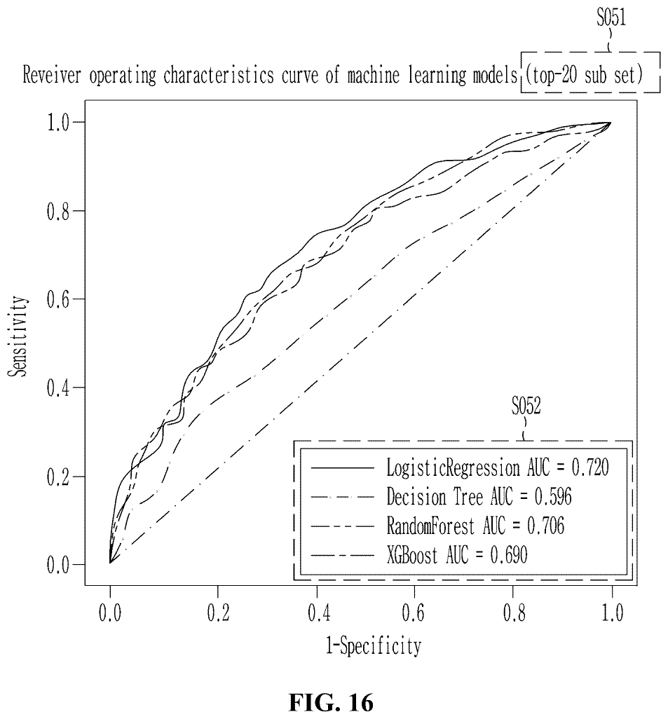

FIG. 16 is a graph showing performance of AI models re-trained by using data of optimum survey questions formed depending on AI models according to an embodiment of the present disclosure.

Referring to FIG. 16, the AI model module 120 may form a data set (top-20 sub set) with the collected optimum survey questions as a test set, evaluate performances of AI models by using the test set, and represent the performances in a graph S051. For example, the AI model module 120 may show the performances of the AI models regarding the optimum survey questions in the graph S051.

According to an embodiment of the present disclosure, the AI model module 120 may determine performances of a plurality of AI models S052 including the 4 types of AI models regarding the optimum survey questions.

According to an embodiment of the present disclosure, the AI model module 120 may verify a performance decrease rate by comparing the graph for the performances regarding the optimum survey questions S051 with the graph for the performances regarding the entire survey questions S041. When the performance decrease rate of the AI models is lower than a number decrease rate (for example, 80.77%) through comparison of the number of the optimum survey questions (for example, 20) with the number of the entire survey questions (for example, 104), the AI model module 120 may determine that the performance decrease rate of the AI models is within a reference range, and thus, determine the optimum survey questions as final survey questions.

According to an embodiment of the present disclosure, since the electronic device 100 may let a user answer optimum survey questions of which the performance decrease rate of the AI models is within a reference range, the user may complete answering within 10 minutes or so, whereas it would generally take about 50 minutes for a user to answer the entire survey questions off-line.

FIGS. 17A and 17B are a diagram showing a decision tree model in which prediction values are branched in forms of AI models according to an embodiment of the present disclosure.

Referring to FIGS. 17A and 17B, the decision tree model may comprise an optimum tree S061 without leaf nodes and leaf nodes S062.

According to an embodiment of the present disclosure, the AI model module 120 may adjust a prediction value by using a prediction value of an AI model and additionally using entropy of a final node corresponding to the AI model.

According to an embodiment of the present disclosure, the AI model module 120 may deduce a path of answer data used for prediction by using the optimum tree S061 and the leaf nodes S062 and verify a leaf node corresponding to the path to adjust a prediction value with the relevant entropy.

According to an embodiment of the present disclosure, the AI model module 120 may make prediction values for answer data of a user and entropies as data to transmit them to the branching recommending module 130.

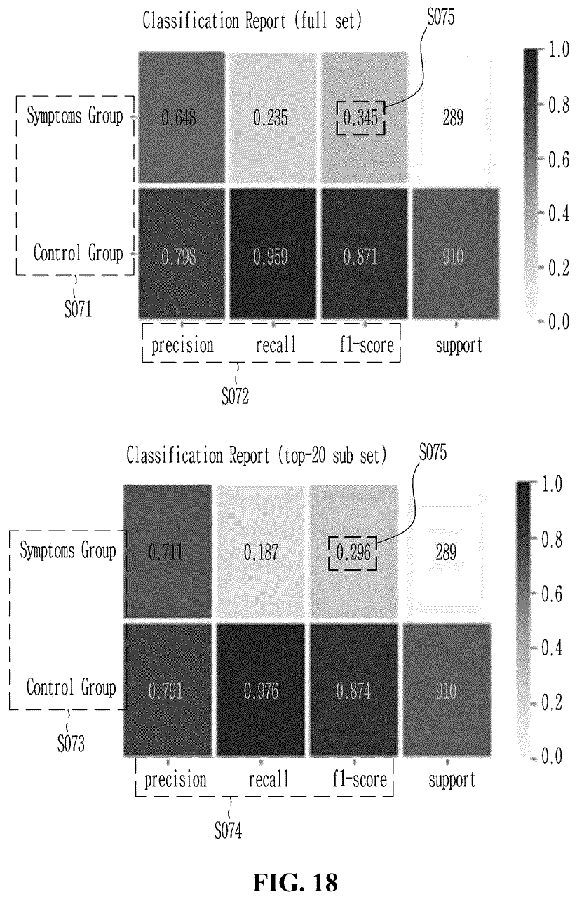

FIG. 18 is a graph showing results of verification of performances of trained AI models according to an embodiment of the present disclosure.

FIG. 18 shows graphs of classification results (classification report) regarding the entire survey questions (full set) and reduced survey questions (top-20 sub set), which are the optimum survey questions.

According to an embodiment of the present disclosure, in each classification result, the AI model module 120 may make binary classification of a normal group and a symptoms group S071, S073 and each group of users may be classified into several groups based on a performance verification method such as precision, recall, and an f1-score S072, S074.

According to an embodiment of the present disclosure, the AI model module 120 may verify values of the f1-scores S075 of the symptoms groups as indexes for prediction. For example, the AI model module 120 may verify that, although an AI model for the reduced survey questions has a lower f1-score than a f1-score of an AI model for the entire survey questions, it has a higher precision value. In this case, the AI model module 120 may induce a more precise prediction of the AI model for the reduced survey questions by collecting some additional questions.

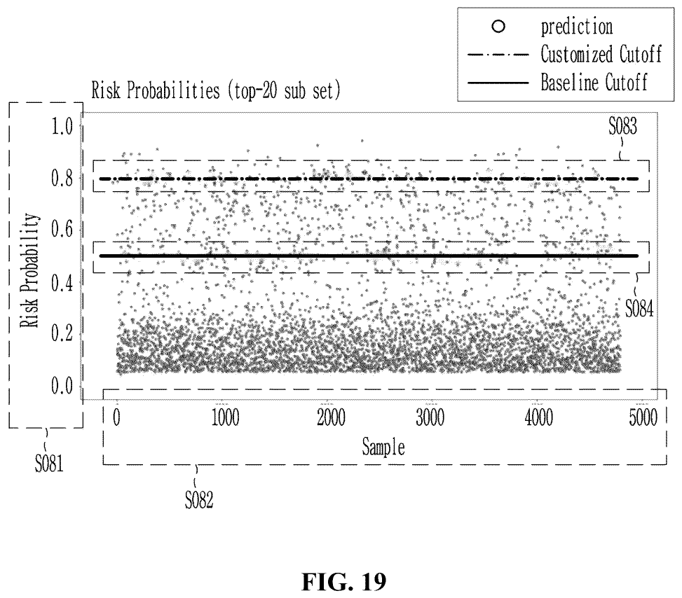

FIG. 19 is a graph for determining an object for a periodic management by using prediction values of a plurality of AI models according to an embodiment of the present disclosure.

Referring to FIG. 19, the object determining module 150 may represent a risk probability of symptoms S081 based on the number of pieces of data of a user S082 in a graph. In the graph, for example, 0.0 may mean 0% and 1.0 may mean 100%.

According to an embodiment of the present disclosure, the object determining module may adjust a threshold point according to an opinion of a psychiatrist and indicate the adjusted threshold point S083 in the graph. In addition, the object determining module may verify a basic determination threshold point S084 of the AI models and indicate the determination threshold point S084 in the graph.

According to an embodiment of the present disclosure, the object determining module may indicate a prediction value for each user in the graph with a dot. When the sensitivity of an AI model exceeds the determination threshold point S084, the sensitivity may be set to be low by setting the adjusted threshold point S083 as the determination threshold point S084 according to a determination of a psychiatrist. Then, the object determining module 150 may periodically perform an operation of setting the determination threshold point S083 and the adjusted threshold point S083 and select an object for which a survey will be conducted in every corresponding period.

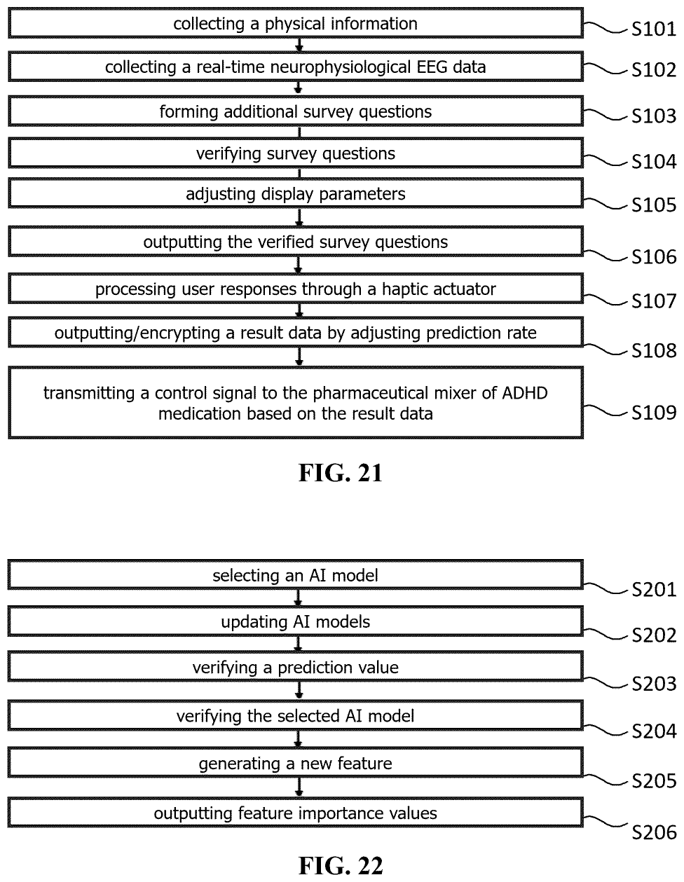

Referring now to FIG. 21, according to an exemplary embodiment, a method for controlling a pharmaceutical mixer of the ADHD medication based on an assessment of a mental health state of an adolescent (assessed by using a survey formed based on an AI) comprises: (S101) an operation of collecting physical information of a user through a pre-installed application, verifying survey questions regarding mental health depending on the physical information of the user, and verifying answers to the verified survey questions inputted by the user through the application; (S102) an operation of collecting EEG signal data and using it to refine survey questions; (S103) an operation of forming additional survey questions for the user after having verified the answers and calculating a prediction rate of an appearance of symptoms of a mental illness regarding the additional survey questions by using AI models; (S104) an operation of verifying a set of survey questions including a plurality of sub-questions depending on the calculated prediction rate of an appearance of symptoms of a mental illness; (S105) an operation of dynamically adjusting display elements based on cognitive load and user interaction; (S106) an operation of outputting the verified set of survey questions through the application; an operation of outputting result data by adjusting the prediction rate of an appearance of symptoms of a mental illness when the set of survey questions is not verified; and an operation of transmitting the outputted result data; an operation of providing haptic feedback to guide user responses; an operation of encrypting data for secure transmission; an operation of selecting an AI model dynamically based on real-time feature analysis; (S106) an operation of outputting the verified set of survey questions through the pre-installed application; (S107) an operation of processing user responses through a haptic actuator, including generation of predetermined adaptive haptic feedback signals based on detected user engagement levels; (S108) an operation of encrypting the result data, wherein the result data is stored in a cloud-based or local encrypted database; and (S109) an operation of transmitting a control signal to the pharmaceutical mixer of the medication for ADHD based on the result data.

Referring now to FIG. 22, according to an exemplary embodiment, the operation of forming additional survey questions (S103) by using AI models may further include: (S201) an operation of selecting an AI model depending on the number of additional survey questions; (S202) an operation of dynamically selecting and updating AI models, wherein the AI model selection is adjusted based on the real-time user response patterns and survey answers; (S203) an operation of verifying a prediction value of the selected AI model regarding the additional survey questions by using the selected AI model; (S204) an operation of verifying the selected AI model according to evaluation indexes preset based on the verified prediction value; (S205) an operation of generating a new feature by means of the above mentioned Equation 1, which is

$${F}_{n e w} = {\sum }_{i = 1}^{N} ( {P}_{i} )$$

$${P}_{i} = {p}_{i} {W}_{i}$$

where F_{new} is a new feature, N is a total number of a plurality of AI models, Pᵢ is an adjusted prediction value of each AI model, pᵢ is the prediction value of each AI model, and Wᵢ is an entropy in a decision tree model and a weighting in other AI models; and (S206) an operation of outputting a set of feature importance values collected by the new features by means of the above mentioned Equation 2, which is

$$SI = \{ {IMP}_{f 1} , {IMP}_{f 2} , … , {IMP}_{fN} \}$$

$${IMP}_{fN} = \vert \frac{( {IMP}_{Af 1} + {IMP}_{Bf 1} + {IMP}_{Cf 1} + … + {IMP}_{Mf 1} )}{{N}_{models}} \vert$$

where SI is a set of collected feature importance values, IMP is an importance value, N is a number of a feature, A-M are identification information of AI models A to M, IMP_{fN} is a final importance value of one feature derived from a plurality of AI models, IMP_{Mf1} is importance values of first features of the AI models, and N_{models} is the number of AI models.

Further, according to an exemplary embodiment, the method may further include: outputting questions regarding age, height, weight, or waist measurement of the user and collecting the answers as physical information of the user by receiving answers to the outputted questions; acquiring the real-time neurophysiological EEG data from the EEG sensor to complement the collected physical information and enhance a predetermined accuracy level of the AI-based mental health predictions; and outputting by using the AI models, among the questions regarding the physical information of the user, survey questions tailored based on both the user-inputted physical information and the real-time neurophysiological EEG data to which answers is obtained within a predetermined time.

According to another exemplary embodiment, the method may further include: confirming survey questions, corresponding to the feature importance values included in the set of calculated feature importance values, as the additional survey questions; and providing haptic feedback via the haptic actuator to indicate validation, progress, or required adjustments during a survey completion, based on the AI-processed importance values. Also, according to another exemplary embodiment, the method may further include outputting the additional questions confirmed in a descending order of feature importance values calculated by equation 2. Further, in another exemplary embodiment, the method may further include: periodically re-verifying the set of survey questions for the user when the prediction rate of the appearance of symptoms of the mental illness exceeds a predetermined value; and selecting the AI model from a plurality of trained models based on real-time data streams, including EEG readings and haptic interaction patterns.

As described above, embodiments according to the present disclosure may be implemented by various means, for example, hardware, firmware, software, or their combination. When it comes to hardware, an embodiment of the present disclosure may be implemented by one or more of application specific integrated circuits (ASICs), digital signal processors (DSPs), digital signal processing devices (DSPDs), programmable logic devices (PLDs), field programmable gate arrays (FPGAs), processors, controllers, micro controllers, microprocessors, and the like.

When it comes to firmware or software, an embodiment of the present disclosure may be implemented in forms of a module, a process, a function, or the like to perform the above-described functions or operations and written in recording media readable by various computer means.

Here, the recording media may include program instructions, data files, data structures, or the like separately or in combination. Program instructions to be written in the recording media may be specifically designed and constructed for the present disclosure or known to persons in the field of computer software to be available to them. For example, the recording media may comprise hardware devices specifically formed to store and execute program instructions, such as magnetic media like hard disks, floppy disks, and magnetic tapes; optical media like compact disk read only memories (CD-ROMs) and digital video disks (DVDs); magneto-optical media like floptical disks, ROMs, RAMs, flash memories, or the like. Examples for program instructions may comprise machine language codes formed by, for example, compilers and high-level language codes executable by computers by using interpreters as well. Hardware devices as described above may be configured to operate as one or more software modules in order to perform the operations of the present disclosure, and vice versa.

In addition, a device or a terminal according to the present disclosure may be driven by instructions to cause one or more processers to execute the above-described functions and processes. For example, such instructions may comprise interpretable instructions like script instructions, such as JavaScript or ECMAScript instructions, executable codes, or other instructions stored in computer readable media. Further, a device according to the present disclosure may be implemented in a distributed form, such as Server Farm, over the network or implemented in a single computer device.

In addition, computer programs (also known as programs, software, software applications, or codes) to be installed in a device according to the present disclosure and to execute a method according to the present disclosure may be written by any types of programming languages including compiled or interpreted languages or innate or procedural languages and may be deployed in any forms including stand-alone programs, components, sub-routines or other units appropriate for being used in a computer environment. Computer programs do not necessarily correspond to files in the file system. A program may be stored in a single file provided to a requested program, in multiple interactive files (for example, files storing one or more modules, subprograms, or portions of code), or in a part of a file that holds other programs or data (for example, one or more scripts stored in a markup language document). A computer program may be deployed to be executed on multiple computers or on one computer, located at a single site or distributed across multiple sites and interconnected by a communications network.

Although respective figures are separately described for the convenience of description, it is also possible to design to implement a new embodiment by combining the embodiments described with respect to the figures. In addition, in the present disclosure, the configurations and methods of the above-described embodiments are not limitedly applied, but all or some of the respective embodiments may be selectively combined for various modifications.

Moreover, although preferred embodiments are illustrated and described in the above, the present disclosure is not limited to the above-described specific embodiments, but can be variously modified by a person skilled in the art, to which the present disclosure pertains, without being beyond the purport of the claims. In addition, such modified embodiments should not be understood to depart from the technological idea or the prospect of the present disclosure.

What is claimed is:

1. A method for controlling a pharmaceutical mixer of a medication for Attention Deficit Hyperactivity Disorder (ADHD) based on an assessment of a mental health state of an adolescent by using a bioelectrical activity data collected by an electroencephalogram (EEG) sensor in response to a survey formed based on an artificial intelligence (AI), wherein a processor and one or more memory devices communicatively coupled to the processor, and the one or more memory devices stores instructions operable when executed by the processor to perform:
collecting a physical information of a user, verifying survey questions regarding a mental health depending on the physical information of the user, and verifying answers to the verified survey questions inputted by the user;
collecting a real-time neurophysiological EEG data via the EEG sensor and integrating a collected EEG data with AI-based survey assessments; and
forming additional survey questions for the user after having the verified answers and calculating a prediction rate of an appearance of symptoms of a mental illness regarding the additional survey questions by using AI models, wherein the forming of the additional survey question comprises:
selecting an AI model depending on a number of the additional survey questions;
updating AI models, wherein the AI model selection is adjusted based on the real-time user response patterns and the verified answers;
verifying a prediction value of the selected AI model regarding the additional survey questions by using the selected AI model;
verifying the selected AI model according to evaluation indexes preset based on the verified prediction value;
generating a new feature by means of equation 1, which is
$${F}_{n e w} = {\sum }_{i = 1}^{N} ( {P}_{i} )$$
$${P}_{i} = {p}_{i} {W}_{i}$$
wherein F_{new} is a new feature, N is a total number of a plurality of AI models, Pᵢ is an adjusted prediction value of each AI model, pᵢ is the prediction value of each AI model, and Wᵢ is an entropy in a decision tree model and a weighting in other AI models,
outputting a set of feature importance values collected by the new features by means of equation 2, which is
$$SI = \{ {IMP}_{f 1} , {IMP}_{f 2} , … , {IMP}_{fN} \}$$
$${IMP}_{fN} = \vert \frac{( {IMP}_{Af 1} + {IMP}_{Bf 1} + {IMP}_{Cf 1} + … + {IMP}_{Mf 1} )}{{N}_{models}} \vert$$
wherein SI is a set of collected feature importance values, IMP is an importance value, N is a number of a feature, A-M are identification information of AI models A to M, IMP_{fN} is a final importance value of one feature derived from a plurality of AI models, IMP_{Mf1} is importance values of first features of the AI models, and N_{models} is the number of AI models,
wherein the one or more memory devices stores instructions operable when executed by the processor to further perform:
verifying a set of survey questions including a plurality of sub-questions depending on the calculated prediction rate of the appearance of symptoms of the mental illness;
adjusting display parameters displayed for the user to predetermined parameters based on AI-driven display adjustments;
outputting the verified set of survey questions;
processing user responses through a haptic actuator, including generation of predetermined adaptive haptic feedback signals based on detected user engagement levels;
outputting a result data by adjusting the prediction rate of the appearance of symptoms of the mental illness when the set of survey questions is not verified;
encrypting the result data, wherein the result data is stored in a cloud-database or a local encrypted database; and
transmitting a control signal to the pharmaceutical mixer of the medication for ADHD based on the result data.

2. The method of claim 1, wherein the one or more memory devices stores instructions operable when executed by the processor to further perform:
outputting questions regarding age, height, weight, or waist measurement of the user and collecting the answers as physical information of the user by receiving answers to the outputted questions; and
acquiring the real-time neurophysiological EEG data from the EEG sensor to complement the collected physical information and enhance a predetermined accuracy level of the AI-based mental health predictions.

3. The method of claim 2, wherein the one or more memory devices stores instructions operable when executed by the processor to further perform:
outputting by using the AI models, among the questions regarding the physical information of the user, survey questions tailored based on both the user-inputted physical information and the real-time neurophysiological EEG data to which answers is obtained within a predetermined time.

4. The method of claim 1, wherein the one or more memory devices stores instructions operable when executed by the processor to further perform:
confirming survey questions, corresponding to the feature importance values included in the set of calculated feature importance values, as the additional survey questions; and
providing haptic feedback via the haptic actuator to indicate validation, progress, or required adjustments during a survey completion, based on the AI-processed importance values.

5. The method of claim 1, wherein the one or more memory devices stores instructions operable when executed by the processor to further perform:
outputting the additional questions confirmed in a descending order of feature importance values calculated by equation 2.

6. The method of claim 1, wherein the one or more memory devices stores instructions operable when executed by the processor to further perform:
periodically re-verifying the set of survey questions for the user when the prediction rate of the appearance of symptoms of the mental illness exceeds a predetermined value; and
selecting the AI model from a plurality of trained models based on real-time data streams, including EEG readings and haptic interaction patterns.

7. An apparatus for controlling a pharmaceutical mixer of a medication for Attention Deficit Hyperactivity Disorder (ADHD) based on an assessment of a mental health state of an adolescent by using a bioelectrical activity data collected by an electroencephalogram (EEG) sensor in response to a survey formed based on an artificial intelligence (AI), the apparatus comprising:
a processor; and
one or more memory devices communicatively coupled to the processor, wherein the one or more memory devices stores instructions operable when executed by the processor to perform:
collecting a physical information of a user, verifying survey questions regarding a mental health depending on the physical information of the user, and verifying answers to the verified survey questions inputted by the user;
collecting a real-time neurophysiological EEG data via the EEG sensor and integrating a collected EEG data with AI-based survey assessments; and
forming additional survey questions for the user after having the verified answers and calculating a prediction rate of an appearance of symptoms of a mental illness regarding the additional survey questions by using AI models, wherein the forming of the additional survey question comprises:
selecting an AI model depending on a number of the additional survey questions;
updating AI models, wherein the AI model selection is adjusted based on the real-time user response patterns and the verified answers;
verifying a prediction value of the selected AI model regarding the additional survey questions by using the selected AI model;
verifying the selected AI model according to evaluation indexes preset based on the verified prediction value;
generating a new feature by means of equation 1, which is
$${F}_{n e w} = {\sum }_{i = 1}^{N} ( {P}_{i} )$$
$${P}_{i} = {p}_{i} {W}_{i}$$
wherein F_{new} is a new feature, N is a total number of a plurality of AI models, Pᵢ is an adjusted prediction value of each AI model, pᵢ is the prediction value of each AI model, and Wᵢ is an entropy in a decision tree model and a weighting in other AI models,
outputting a set of feature importance values collected by the new features by means of equation 2, which is
$$SI = \{ {IMP}_{f 1} , {IMP}_{f 2} , … , {IMP}_{fN} \}$$
$${IMP}_{fN} = \vert \frac{( {IMP}_{Af 1} + {IMP}_{Bf 1} + {IMP}_{Cf 1} + … + {IMP}_{Mf 1} )}{{N}_{models}} \vert$$
wherein SI is a set of collected feature importance values, IMP is an importance value, N is a number of a feature, A-M are identification information of AI models A to M, IMP_{fN} is a final importance value of one feature derived from a plurality of AI models, IMP_{Mf1} is importance values of first features of the AI models, and N_{models} is the number of AI models,
wherein the one or more memory devices stores instructions operable when executed by the processor to further perform:
verifying a set of survey questions including a plurality of sub-questions depending on the calculated prediction rate of the appearance of symptoms of the mental illness;
adjusting display parameters displayed for the user to predetermined parameters based on AI-driven display adjustments;
outputting the verified set of survey questions;
processing user responses through a haptic actuator, including generation of predetermined adaptive haptic feedback signals based on detected user engagement levels;
outputting a result data by adjusting the prediction rate of the appearance of symptoms of the mental illness when the set of survey questions is not verified;
encrypting the result data, wherein the result data is stored in a cloud-database or a local encrypted database; and
transmitting a control signal to the pharmaceutical mixer of the medication for ADHD based on the result data.

8. The apparatus of claim 7, wherein the one or more memory devices stores instructions operable when executed by the processor to further perform:
outputting questions regarding age, height, weight, or waist measurement of the user and collecting the answers as physical information of the user by receiving answers to the outputted questions; and
acquiring the real-time neurophysiological EEG data from the EEG sensor to complement the collected physical information and enhance a predetermined accuracy level of the AI-based mental health predictions.

9. The apparatus of claim 8, wherein the one or more memory devices stores instructions operable when executed by the processor to further perform:
outputting by using the AI models, among the questions regarding the physical information of the user, survey questions tailored based on both the user-inputted physical information and the real-time neurophysiological EEG data to which answers is obtained within a predetermined time.

10. The apparatus of claim 7, wherein the one or more memory devices stores instructions operable when executed by the processor to further perform:
confirming survey questions, corresponding to the feature importance values included in the set of calculated feature importance values, as the additional survey questions; and
providing haptic feedback via the haptic actuator to indicate validation, progress, or required adjustments during a survey completion, based on the AI-processed importance values.

11. The apparatus of claim 7, wherein the one or more memory devices stores instructions operable when executed by the processor to further perform:
outputting the additional questions confirmed in a descending order of feature importance values calculated by equation 2.

12. The apparatus of claim 7, wherein the one or more memory devices stores instructions operable when executed by the processor to further perform:
periodically re-verifying the set of survey questions for the user when the prediction rate of the appearance of symptoms of the mental illness exceeds a predetermined value; and
selecting the AI model from a plurality of trained models based on real-time data streams, including EEG readings and haptic interaction patterns.
